(19) United States
(12) Patent Application Publication
(10) Pub. No.: US 2007/0004651 A1
(43) Pub. Date: Jan. 4, 2007

(54) Active ingredient combinations of one or more isoflavonoids and carnitine and/or one or more acyl-carnitines

(75) Inventors: Uwe Schoenrock, Nahe (DE); Volker Schreiner, Hamburg (DE); Franz Staeb, Echem (DE); Thomas Doering, Dormagen (DE); Helga Biergiesser, Reinbek (DE); Thomas Blatt, Wedel (DE); Stefan Gallinat, Wedel (DE); Kirsten Ventzke, Emden (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/295,566

(22) Filed: Dec. 7, 2005

(30) Foreign Application Priority Data
Dec. 8, 2004 (DE) 10 2004 060 314.6

(51) Int. Cl. A61K 31/7048; A61K 31/353; A61K 31/35; A61K 31/22; A61K 31/205

(52) U.S. Cl. 514/27; 514/456; 514/546; 514/554

(57) ABSTRACT

Active ingredient combinations of (a) one or more isoflavonoids and (b) carnitine and/or one or more acylcarnitines.

The present invention relates to cosmetic or dermatological preparations containing active ingredients for the care and protection of the skin, in particular of sensitive skin, and also quite particularly prominently of skin that is aging or has been aged due to intrinsic and/or extrinsic factors, as well as the use of such active ingredients and combinations of such active ingredients in the field of cosmetic and dermatological skin care.

Skin care is to be understood primarily to mean that the natural function of the skin as a barrier against environmental influences (e.g. dirt, chemicals, microorganisms) and against the loss of endogenous substances (e.g. water, natural fats, electrolytes) is strengthened or restored.

If this function is impaired, the result can be increased absorption of toxic or allergenic substances or attack by microorganisms and as a result, toxic or allergic skin reactions.

In old skin, for example, the regenerative renewal is slowed, whereby in particular the water-binding capacity of the corneum decreases. It therefore becomes inflexible, dry, and cracked (“physiologically” dry skin). The result is barrier damage. The skin becomes susceptible to adverse environmental influences such as the invasion of microorganisms, toxins, and allergens. This can even result in toxic or allergic skin reactions.

In pathologically dry and sensitive skin, barrier damage is present a priori. Epidermal intercellular lipids are formed incorrectly or in insufficient quantity or composition. The consequence is an increased permeability of the corneum and inadequate protection of the skin from loss of hygroscopic substances and water.

The barrier action of the skin can be quantified by determining the transepidermal water loss (TEWL). This is the evaporation of water from the interior of the body without the inclusion of water loss due to sweating. The determination of the TEWL value has proved to be extraordinarily informative and can be used to diagnose cracked or chapped skin, to determine the compatibility of surfactants with different chemical structures, and the like.

The proportion of water in the uppermost dermal layer is of the greatest importance for the beauty and good appearance of the skin. It can be favorably influenced to a limited extent by introducing moisture regulators.

Anionic surfactants, which are generally constituents of cleaning preparations, can raise the pH in the corneum for a long time, which sharply impedes regenerative processes that serve to restore and renew the barrier function of the skin. In this case a new, frequently very unfavorable equilibrium state is established in the corneum between regeneration and the loss of essential substances through regular extraction, which equilibrium state decisively impairs the outer appearance of the skin and the physiological functioning of the corneum.

Even a simple water bath, without the addition of surfactants, results first in a swelling of the corneum of the skin, whereby the degree of this swelling is dependent for example on the duration of the bath and its temperature. At the same time water-soluble substances, e.g. water-soluble dirt constituents, but also endogenous skin substances that are responsible for the water-binding capacity of the corneum, are washed off or out. In addition, sebaceous matter is dissolved and washed out to a certain extent through surface-active endogenous skin substances. After an initial swelling, this causes a subsequent distinct drying of the skin, which can be further increased by detersive additives.

In healthy skin these processes are generally unimportant, since the protective mechanisms of the skin can easily compensate for such slight disturbances of the upper dermal layers. But already in the case of nonpathological deviations from the normal status, e.g. due to environmentally caused abrasion damage or irritations, light damage, senile skin, etc., the protective mechanism of the skin surface is disturbed. Under certain circumstances it is then no longer capable of fulfilling its task by itself and must be regenerated through external measures.

Moreover it is known that the lipid composition and amount of the corneum of the pathologically changed, dry, and the dry but not diseased skin of younger and older humans deviates from the normal state found in the healthy, normally hydrated skin of a group of the same age. Thereby the changes in the lipid pattern of the very dry, noneczematous skin of patients with atopic eczema represent an extreme case of the deviations that are found in the dry skin of humans with healthy skin.

These deviations relate thereby quite particularly to the ceramides, whose amount is sharply reduced and in addition have a different composition. The deficit in ceramides 1 and 3 is particularly striking thereby, whereby in particular it is known for ceramide 1 that it particularly increases the arrangement of the lipids in the intercellular membrane systems.

Detrimental changes in the lipid membranes of the above-described type are possibly based on faulty control of lipid biosynthesis and likewise increase the transepidermal water loss in the final analysis. A long-lasting barrier weakness again makes the per se healthy skin more sensitive and in individual cases can contribute to the occurrence of eczematous processes in the diseased skin.

The effect of salves and creams on the barrier function and hydration of the corneum is not as a rule to restore or strengthen the physical-chemical properties of the lamellae made of intercellular lipids. A considerable partial effect is based on the simple covering of the treated cutaneous areas and the resulting damming of water in the corneum below them. Hygroscopic substances applied at the same time bind the water, resulting in a measurable increase in the water content in the corneum. However, this purely physical barrier can be removed again relatively easily. After the product has been discontinued, the skin returns very rapidly again to the state before the start of treatment. Moreover with regular treatment the skin care effect can diminish, so that finally even during the treatment, the status quo is again reached. With certain products the state of the skin possibly deteriorates temporarily after the discontinuation. A lasting product effect is therefore as a rule not achieved or only achieved to a limited extent.

In order to support the deficient skin in its natural regeneration and to strengthen its physiological function, recently intercellular lipid mixtures, are increasingly being added to topical preparations, which mixtures are intended to be used by the skin to regenerate the natural barrier. However these lipids, but particularly the ceramides, are very expensive raw materials. In addition their effect is usually very much less than had been hoped for.

Thus it was an object of the present invention to find ways to avoid the disadvantages of the prior art. In particular the effect of the skin care products was to be physiological, rapid, and lasting.

Skin care in the sense of the present invention is primarily to be understood to mean that the natural function of the skin as a barrier against environmental influences (e.g. dirt, chemicals, microorganisms) and against the loss of endogenous substances (e.g. water, lipids, electrolytes) is to be strengthened or restored.

Products for the care, treatment, and cleaning of dry and stressed skin are per se known. However their contribution to the regeneration of a physiologically intact, hydrated, and smooth corneum is limited in extent and time.

The action of salves and creams on the barrier function and the hydration of the corneum is based essentially on the covering (occlusion) of the treated cutaneous areas. The salve or cream as it were represents a (second) artificial barrier that is intended to prevent the water loss of the skin. This physical barrier can accordingly be removed easily—for example with cleansing agents—as a result of which the original, impaired state is again reached. Moreover the skin care effect can diminish with regular treatment. After the product use has been discontinued, the skin very quickly returns to the state before the start of treatment. With certain products the state of the skin possibly even deteriorates temporarily. A lasting product effect is therefore as a rule not achieved or only achieved to a limited extent.

The effect of some pharmaceutical preparations on the barrier function of the skin is even a selective barrier damage that is intended to enable active substances to penetrate into or through the skin into the body. An impaired appearance of the skin as a side-effect thereby is somewhat regarded as an acceptable consequence.

The effect of skin care cleansing products is essentially an efficient fat replacement with sebum lipid-like substances. The damage to the corneum barrier can be further limited by the simultaneous reduction in the surfactant content of such preparations.

However, the prior art is lacking in preparations that have a favorable influence on the barrier function and the hydration of the corneum and that strengthen or even restore the physical-chemical properties of the corneum and in particular of the lamellae made of intercellular lipids.

It was therefore an object of the present invention to eliminate the disadvantages of the prior art. In particular, skin care preparations and preparations for cleaning the skin were to be made available that maintain or restore the barrier properties of the skin, especially when the natural regeneration of the skin is insufficient. They are furthermore intended to be suitable for the treatment and prophylaxis of secondary damage of the skin drying, for example fissures or inflammatory or allergic processes, or neurodermatitis. It was also an object of the present invention to make available stable skin care cosmetic and/or dermatological agents that protect the skin from environmental influences such as sun and wind. In particular the action of the preparations was intended to be physiological, rapid, and lasting.

In a further preferred embodiment, the present invention relates to cosmetic and dermatological preparations for the prophylaxis and treatment of cosmetic or dermatological skin changes such as e.g. undesired pigmentation, for example local hyper- and abnormal pigmentation (e.g. liver spots, freckles), but also for the purely cosmetic lightening of larger skin areas that are per se completely appropriately pigmented for the individual skin type.

The melanocytes, which depending on the skin type are to be found as pigment-forming cells occurring either individually or more or less clustered in the lowest layer of the epidermis, the stratum basale, in addition to the basal cells, are responsible for the pigmentation of the skin. Melanocytes contain melanosomes as characteristic cell organelles, which form increased amounts of melanin when excited by UV radiation. The melanin is transported into the keratinocytes and causes a more or less strongly pronounced brownish or brown skin color.

Melanin is formed as the final stage of an oxidative process in which tyrosine is finally converted into melanin under the mediation of the enzyme tyrosinase via 3,4-dihydroxyphenylalanine (dopa), dopa-quinone, leucodopachrome, dopachrome, 5,6-dihydroxyindole, and indole-5,6-quinone.

Problems with hyperpigmentation of the skin have multiple causes or are side-effects of many biological processes, e.g. UV radiation (e.g. freckles, Ephelides), genetic disposition, abnormal pigmentation of the skin during wound healing or wound scarring or skin aging (e.g. Lentigines seniles).

Active substances and preparations are known that counteract the skin pigmentation. In practical use these are essentially preparations based on hydroquinone, which on the one hand do not show an effect until after several weeks of use, and on the other hand their excessively long use gives cause for concern for toxicological reasons. The inhibition of the tyrosinase with substances such as kojic acid, ascorbic acid, and azelaic acid and their derivatives is also common, but has cosmetic and dermatological disadvantages.

It was also an object of the present invention to remedy these problems.

A goal of skin care is also to compensate for the fat- and water loss of the skin caused by daily washing. This is important in particular when the natural regeneration capacity is insufficient. Moreover skin care products are intended to protect against environmental influences, especially sun and wind, and to delay skin aging.

The chronological skin aging is caused e.g. by endogenous, genetically determined factors. Aging causes e.g. the following structural damage and dysfunction in the epidermis and dermis, which can also fall under the heading of “senile xerosis”:
a) dryness, roughness, and the formation of dryness wrinkles,
b) itching, and
c) decreased fat replacement by means of sebaceous glands (e.g. after washing).

Exogenous factors such as UV light and chemical noxae can have a cumulative effect and e.g. accelerate or supplement the endogenous aging processes. E.g. the following structural damage and dysfunction in the skin are caused in particular by exogenous factors in the epidermis and dermis, which exceed the extent and quality of the damage in chronological aging:
d) visible vasodilation (telangiectasis, erythema),
e) flabbiness and formation of folds,
f) local hyper-, hypo-, and abnormal pigmentation (e.g. age spots) and
g) increased susceptibility to mechanical stress (e.g. cracking).

The present invention relates in particular to products for the care of naturally aged skin, as well as for the treatment of secondary damage of light aging, in particular the phenomena listed under a) to g).

Products for the care of aged skin are per se known. They contain e.g. retinoids (vitamin A acid and/or its derivatives) or vitamin A and/or its derivatives. However, their effect on the structural damage is limited in extent. Moreover in the product development, considerable difficulties arise in stabilizing the active substances adequately against oxidative decomposition. Moreover, the use of products containing vitamin A acid often causes strong erythematous skin irritations. Retinoids can therefore only be used in low concentrations.

In particular the present invention relates to cosmetic preparations with an efficacious protection from detrimental oxidation processes in the skin, but also for the protection of cosmetic preparations themselves or for the protection of the constituents of cosmetic preparations from detrimental oxidation processes.

The present invention also relates to antioxidants, preferably those that are used in skin care cosmetic or dermatological preparations. In particular the invention also relates to cosmetic and dermatological preparations containing such antioxidants. In a preferred embodiment, the present invention relates to cosmetic and dermatological preparations for the prophylaxis and treatment of cosmetic or dermatological skin changes such as e.g. skin aging, in particular skin aging caused by oxidative processes.

Furthermore the present invention relates to active ingredients and preparations containing such active ingredients, for the cosmetic and dermatological treatment or prophylaxis of erythematous, inflammatory, allergic, or autoimmune phenomena, in particular dermatoses.

In a further advantageous form of embodiment the present invention relates to active ingredient combinations and preparations that serve for the prophylaxis and treatment of light-sensitive skin, in particular photodermatoses.

The damaging effect of the ultraviolet region of solar radiation on the skin is generally known. Whereas rays with a wavelength below 290 nm (the so-called UVC region) are absorbed by the ozone layer in the earth's atmosphere, rays in the region between 290 nm and 320 nm, the so-called UVB region, cause an erythema, a simple sunburn, or even more or less severe burns.

The narrower region around 308 nm is given as a maximum of erythema effectiveness of sunlight.

Numerous compounds are known for protection against UVB radiation; they are derivatives of 3-benzylidenecamphor, 4-aminobenzoic acid, cinnamic acid, salicylic acid, benzophenone, and 2-phenylbenzimidazole.

It is important also to have filter substances available for the region between about 320 nm and about 400 nm, the so-called UVA region, since these rays can cause reactions in light-sensitive skin. It has been proved that UVA radiation leads to a damaging of the elastic and collagen fibers of the connective tissue, which causes the skin to age prematurely, and that UVA radiation is to be considered the cause of numerous phototoxic and photoallergic reactions. The damaging effect of UVB radiation can be intensified by UVA radiation.

Certain derivatives of dibenzoylmethane are therefore used for protection against the rays in the UVA region, but their photostability (Int. J. Cosm. Science 10, 53 (1988)), is inadequate.

However, the UV radiation can also lead to photochemical reactions, whereby the photochemical reaction products then interfere in the skin metabolism.

Such photochemical reaction products are primarily radical compounds, for example hydroxyl radicals, singlet oxygen. Undefined radical photoproducts that are formed in the skin itself can also display uncontrolled secondary reactions due to their high reactivity. However, singlet oxygen, a non-radical excited state of the oxygen molecule, can also occur with UV radiation, as can short-lived epoxides and many others. Singlet oxygen, for example, is distinguished from the normally present triplet oxygen (radical normal state) by elevated reactivity. However, excited, reactive (radical) triplet states of the oxygen molecule also exist.

Furthermore UV radiation is counted as ionizing radiation. There is therefore the risk that ionic species are also formed during UV exposure that then are capable of interfering oxidatively in the biochemical processes.

In order to avert these reactions, additional antioxidants and/or free radical scavengers can be incorporated into the cosmetic or dermatological formulations.

It has already been suggested to use vitamin E, a substance with known antioxidative action, in sunscreen formulations, but here too the effect achieved falls far short of what was hoped.

It was therefore also an object of the invention to create cosmetic, dermatological and pharmaceutical active ingredients and preparations as well as sunscreen formulations, which serve for the prophylaxis and treatment of light-sensitive skin, in particular photodermatoses, preferably PLD.

Further designations for polymorphous photodermatosis are PLD, PLE, Mallorca acne, and a number of other designations as given in the literature (e.g. A. Voelckel et al., Zentralblatt Haut- und Geschlechtskrankheiten (1989), 156, p. 2).

Antioxidants are chiefly used as protective substances against the deterioration of the preparations containing them. However, it is known that undesired oxidation processes can also occur in human and animal skin. Such processes play a considerable role in skin aging.

In the article “Skin Diseases Associated with Oxidative Injury” in “Oxidative Stress in Dermatology”, p. 323 ff. (Marcel Decker Inc., New York, Basel, Hong Kong, Editors: Jürgen Fuchs, Frankfurt, and Lester Packer, Berkeley, Calif.), oxidative injuries of the skin and their precise causes are listed.

Antioxidants and/or free radical scavengers can additionally be incorporated into cosmetic or dermatological formulations based on averting such reactions.

Some antioxidants and free radical scavengers are indeed known. Thus it has already been suggested in U.S. Pat. No. 4,144,325 and 4,248,861, as well as from numerous other documents, to use vitamin E, a substance with known antioxidative action in sunscreen formulations, but here too the effect achieved falls far short of what was hoped.

It was thus an object of the present invention to find ways of avoiding the disadvantages of the prior art. In particular the action of the repair of the damages associated with the endogenous, chronological, and exogenous skin aging and the prophylaxis are to be durable, lasting, and without the risk of side-effects.

According to the invention the problems of the prior art are eliminated by means of active ingredient combinations of
(a) one or more isoflavonoids and
(b) carnitine and/or one or more acylcarnitines.

The active ingredient combinations according to the invention or cosmetic or dermatological preparations containing such active ingredients are completely satisfactory preparations in every respect. It could not be predicted by those skilled in the art that the preparations according to the invention
maintain or restore the barrier properties of the skin better,
counteract the skin drying better,
have a better effect on dyschromia,
have a better effect on skin aging, and
protect the skin better from environmental influences than the preparations of the prior art.

When the active ingredient combinations used according to the invention or cosmetic or topical dermatological preparations with an effective content of active ingredient combinations used according to the invention are applied, surprisingly an efficacious treatment, but also a prophylaxis
of deficient, sensitive, or hypoactive skin conditions or of deficient, sensitive, or hypoactive states of integumentary appendages,
of phenomena of premature aging of the skin (e.g. folds, age spots, telangiectases) and/or of the integumentary appendages,
of environmentally caused (smoking, smog, reactive oxygen species, free radicals) and in particular light-caused adverse changes of the skin and of the integumentary appendages,
of light-caused skin damage,
of dyschromia,
of itching,
of dry skin conditions and corneum barrier impairment,
of hair loss and for improved hair growth,
of inflammatory skin conditions as well as atopic dermatitis, seborrheic dermatitis, polymorphous photodermatosis, psoriasis, vitiligo
   are possible. However, the active ingredients according to the invention or cosmetic or topical dermatological preparations with an effective content of active ingredient according to the invention also serve surprisingly
to soothe sensitive or irritated skin
to stimulate the synthesis of collagen, hyaluronic acid, elastin
to stimulate the ceramide synthesis of the skin
to stimulate the intracellular DNA synthesis, in particular in deficient or hypoactive skin conditions
to increase the cell renewal and regeneration of the skin
to increase the endogenous skin protection and repair mechanisms (for example for dysfunctional enzymes, DNA, lipids, proteins)
for the pre- and post-treatment with topical use of laser- and abrasion treatments that serve e.g. to reduce skin folds and scars, in order to counteract the resulting skin irritations and to promote the regeneration processes in the injured skin.

Therefore the use of active ingredient combinations of
(a) one or more isoflavones and
(b) carnitine and/or one or more acylcarnitines
   for the prophylaxis and treatment of inflammatory skin conditions—also atopic dermatitis—and/or for skin protection in sensitively determined dry skin is also according to the invention.

Furthermore the use of active ingredient combinations of
(a) one or more isoflavonoidss and
(b) carnitine and/or one or more acylcarnitines
   for the production of cosmetic or dermatological preparations for the treatment and/or prophylaxis of dyschromia is also according to the invention.

Furthermore the use of active ingredient combinations of
(a) one or more isoflavonoids and
(b) carnitine and/or one or more acylcarnitines
   for the production of cosmetic or dermatological preparations for the treatment and/or prophylaxis of the symptoms of intrinsic and/or extrinsic skin aging as well as for the treatment and prophylaxis of the harmful effects of ultraviolet radiation on the skin is also according to the invention.

Furthermore the use of active ingredient combinations of
(a) one or more isoflavonoids and
(b) carnitine and/or one or more acylcarnitines
   for the production of cosmetic or dermatological preparations for increasing the ceramide biosynthesis is also according to the invention.

Furthermore the use of active ingredient combinations of
(a) one or more isoflavonoids and
(b) carnitine and/or one or more acylcarnitines
   for the production of cosmetic or dermatological preparations for strengthening the barrier function of the skin is also according to the invention.

The active ingredient combinations according to the invention have a synergistic effect with respect to the individual components in all these uses.

L-carnitine [3-hydroxy4-(trimethylammonio)butyric acid betaine] has the structural formula 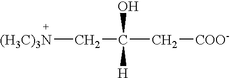
(empirical formula C₇H₁₅NO₃).

The L-form of carnitine is widely distributed in animal tissues, in particular the striated muscles. In the fatty acid metabolism, it serves as a transfer agent for acyl groups through the mitochondrial membrane. These groups are transferred by means of an acyl transferase of acyl-coenzyme-A to the hydroxy group of the L-carnitine. The transport of L-carnitine and acyl-L-carnitine through the membrane takes place through the mediation of a transport protein (translocase). Both enantiomers (D- and L-form) are advantageous to use in the sense of the present invention. It can also be advantageous to use any desired enantiomer mixtures, for example a racemate of D- and L-form.

According to the invention acylcarnitines are selected from the group of substances of the following general structural formula 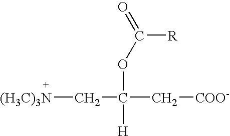
where R is selected from the group of branched and unbranched alkyl radicals with up to 10 carbon atoms. Propionylcarnitine is preferred, and, quite particularly preferred, acetylcarnitine. Both enantiomers (D- and L-form) are advantageously to be used in the sense of the present invention. It can also be advantageous here to use any desired enantiomer mixtures, for example a racemate of D- and L-form.

Advantageously the preparations according to the invention contain 0.001-10% by wt of carnitine and/or one or more acylcarnitines, relative to the total weight of the preparations.

Isoflavones are the group of plant pigments, mostly yellowish in color and derived from isoflavone, which counts as belonging to the flavonoids and is sometimes also called isoflavonoids. The unsubstituted parent substance, the actual isoflavone (3-phenylchromone, 3-phenyl-4H-1-benzopyran-4-one) occurs in clover species.

Some better-known isoflavones are daidzein (4′,7-dihydroxyisoflavone), as 7-O-glucoside daidzin in soybean flour; genistein (4′,5,7-trihydroxyisoflavone) from soybeans and red clover; prunetin (4′,5-dihydroxy-7-methoxyisoflavone) from the bark of plum trees; biochanin A (5,7-dihydroxy-4′-methoxyisoflavone) from chickpeas, red clover and other clover species; orobol (3′,4′,5,7-tetrahydroxyisoflavone; santal (3′,4′,5-trihydroxy-7-methoxyisoflavone) from sandalwood, redwood, and other woods; pratensein (3′,5,7-trihydroxy-4′-methoxyisoflavone) from fresh red clover. Some of these isoflavones, which occur in clover species and leguminous plants such as lucerne, exhibit estrogenic activity in grazing animals and may possibly lead to reproductive disorders in these animals.

Substitution charts of some naturally occurring isoflavones are listed below:

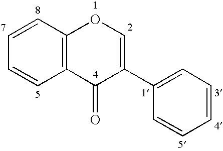

| | 5 | 7 | 3′ | 4′ | CAS No. |
|---|---|---|---|---|---|
| Isoflavone | H | H | H | H | 574-12-9 |
| Daidzein | H | OH | H | OH | 486-68-8 |
| Genistein | OH | OH | H | OH | 446-72-0 |
| Genistin | OH | OH | H | OH | 446-72-0 |
| Prunetin | OH | OCH₃ | H | OH | 552-59-0 |
| Biochanin A | OH | OH | H | OCH₃ | 491-80-5 |
| Orobol | OH | OH | OH | OH | 480-23-9 |
| Santal | OH | OCH₃ | OH | OH | 529-60-2 |
| Pratensein | OH | OH | OH | OCH₃ | 2284-31-3 |

The isoflavones listed in the above table are suitable and preferred as isoflavones to be used according to the invention.

Of these, genistein in turn is particularly advantageous.

Genistein, 5,7-dihydroxy-3-(4-hydroxyphenyl)-4H-1-benzopyran4-one, 4′,5,7-trihydroxyisoflavone, also called differenol A, prunetol, and sophoricol, has the following structure:

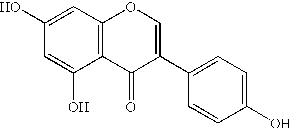

Genistein is a secondary metabolite from plants (legumes plants, papilionoids, Rosaceae), but has also been found in cultures of microorganisms (Actinomycetes, Aspergillus, Mycobacteria). Weakly estrogenic and antibacterial effects have been described.

According to the invention it is preferred to use isoflavone extracts recovered from soybean.

In a form of embodiment particularly preferred according to the invention, the isoflavone extract contains:

| | |
|---|---|
| Glycitein | 0.18% |
| Glycitin | 1.68% |
| Malonyl glycitin | 0.22% |
| Acetyl glycitin | 1.22% |
| Daidzein | 0.08% |
| Daidzin | 3.28% |
| Malonyl daidzein | 0.03% |
| Acetyl daidzin | 2.27% |
| Genistein | 0.03% |
| Genistin | 0.84% |
| Acetyl genistin | 0.70% |
| Malonyl genistin | 0.03% |

The individual concentrations can thereby naturally deviate from the average values given by up to 50%.

In addition to isoflavones, the isoflavone extract can also contain i.a. proteins, saponins, fats, carbohydrates, sugar, and oligosaccharides.

Advantageous isoflavone extracts according to the invention are characterized in that the isoflavone extract in powder form is present such that at least 65% by wt of the particles feature a grain size of less than 30 μm. It is thereby preferred according to the invention if at least 70% of the particles feature a grain size of less than 30 μm.

It is preferred according to the invention that isoflavone extracts can be used that are declared according to INCI with soybean (glycine soya) germ extract.

Isoflavone extracts preferred according to the invention are for example those obtainable from Lucas Meyer Cosmetics S.A. under the trade names “Isoflavones 150” and “Isoflavones Micro”.

Preferably cosmetic or dermatological preparations according to the invention contain 0.001-10% by wt, particularly preferred 0.01-1% by wt, of one or more isoflavones, relative to the total composition of the preparations.

Preferably the weight ratios of one or more isoflavones to carnitine and/or to one or more acylcarnitines in the active ingredient combinations and preparations containing such active ingredient combinations are selected from the range of 50:1 to 1:50, advantageously from the range of 10:1 to 1:50, in particular advantageously 2:1 to 1:2.

In particular it is advantageous thereby if these extracts are additionally characterized by a content of saponins, since the effects to be achieved are particularly pronounced in this case. The content of saponins should thereby lie in a concentration range of 0.001-2% by wt, preferably 0.02-0.04% by wt, relative to the total composition of the cosmetic preparation.

An advantageous embodiment of the present invention thereby can be that the isoflavonoid or isoflavonoids is(are) used as an isoflavonoid-containing extract in the form of a soybean extract that in addition to isoflavonoids, preferably genistein, contains 5-20% by wt of saponins, particularly advantageously 10-18% by wt of saponins, in each case relative to the total weight of the extract.

It is in particular extremely advantageous according to the invention to use the active substance combination or cosmetic or topical dermatological preparations used according to the invention with an effective content of active substance combination used according to the invention for the cosmetic or dermatological treatment or prophylaxis of undesired skin conditions.

According to the invention customary antioxidants can be used preparations that contain the active ingredient combinations according to the invention.

The antioxidants are advantageously selected from the group consisting of amino acids (e.g. glycine, histidine, tyrosine, tryptophan) and their derivatives, imidazoles (e.g. urocanic acid) and their derivatives, peptides such as D,L-carnosine, D-carnosine, L-carnosine, and their derivatives (e.g. anserine), carotenoids, carotenes (e.g. α-carotene, β-carotene, lycopene) and their derivatives, aurothioglucose, propylthiouracil and other thiols (e.g. thioredoxin, glutathione, cysteine, cystine, cystamine and their glycosyl-, N-acetyl-, methyl-, ethyl-, propyl-, amyl-, butyl- and lauryl-, palmitoyl-, oleyl-, y-linoleyl-, cholesteryl-, and glyceryl esters) as well as their salts, dilauryl thiodipropionate, distearyl thiodipropionate, thiodipropionic acid and their derivatives (esters, ethers, peptides, lipids, nucleotides, nucleosides, and salts) as well as sulfoximine compounds (e.g. buthionine sulfoximines, homocysteine sulfoximine, buthionine sulfones, penta-, hexa-, heptathionine sulfoximine) in very small compatible dosages (e.g. pmol to μmol/kg), furthermore (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and their derivatives, unsaturated fatty acids and their derivatives (e.g. γ-linolenic acid, linolic acid, oleic acid), folic acid and its derivatives, alanine diacetic acid, flavonoids, polyphenols, catechinic acids, vitamin C and derivatives (e.g. ascorbyl palmitate, Mg-ascorbyl phosphate, ascorbyl acetate), tocopherols and derivatives (e.g. vitamin E acetate), as well as coniferyl benzoate of the benzoin resin, rutinic acid and its derivatives, ferulic acid and its derivatives, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (e.g. ZnO, ZnSO₄), selenium and its derivatives (e.g. selenium methionine), stilbenes and their derivatives (e.g. stilbene oxide, trans-stilbene oxide), and the derivatives that are suitable according to the invention (salts, esters, ethers, sugars, nucleotides, nucleosides, peptides, and lipids) of these named active substances.

The amount of the antioxidants (one or more compounds) in the preparations is preferably 0.001 to 30% by wt, particularly preferred 0.05-20% by wt, in particular 1-10% by wt, relative to the total weight of the preparation.

The prophylaxis or the cosmetic or dermatological treatment with the active substance used according to the invention or with the cosmetic or topical dermatological preparations with an effective content of active ingredient used according to the invention takes place in the customary manner, such that the active substance used according to the invention or the cosmetic or topical dermatological preparations with an effective content of active substance used according to the invention is applied to the affected cutaneous areas.

Advantageously the active ingredient used according to the invention can be incorporated into customary cosmetic and dermatological preparations, which can be present in various forms. Thus they can represent e.g. a solution, an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or lipodispersion, a gel, a solid stick, or an aerosol.

Emulsions according to the invention in the sense of the present invention, e.g. in the form of a cream, a lotion, a cosmetic milk, are advantageous and contain e.g. fats, oils, waxes, and/or other adipoids, as well as water and one or more emulsifiers, as are customarily used for such a type of formulation.

It is also possible and advantageous in the sense of the present invention to incorporate the active ingredient used according to the invention into aqueous systems or surfactant preparations to clean the skin and hair.

It is of course known to those skilled in the art that high-quality cosmetic compositions are generally inconceivable without the customary auxiliaries and additives. The cosmetic preparations according to the invention can therefore contain cosmetic auxiliaries as are customarily used in such preparations, e.g. preservatives, bactericides, deodorizing substances, antiperspirants, insect repellents, vitamins, agents for the prevention of foaming, dyes, pigments with coloring effect, thickeners, softening substances, moistening and/or moisturizing substances, fats, oils, waxes, or other customary constituents of a cosmetic formulation such as alcohols, polyols, polymers, foam stabilizers, electrolytes, organic solvents, or silicone derivatives.

Mutatis mutandis, corresponding requirements apply to the formulation of medicinal preparations.

Medicinal topical compositions in the sense of the present invention as a rule contain one or more medicinal drugs in an effective concentration. For the sake of simplicity, reference is made to the legal provisions of the Federal Republic of Germany (e.g. Cosmetic Regulations, Food and Drug Law) for a clear distinction between cosmetic and medicinal use and corresponding products.

The preparations according to the invention advantageously, although it is not mandatory, furthermore contain substances that absorb UV radiation in the UV-A and/or UV-B region, whereby the total amount of the filter substances is e.g. 0.1 to 30% by wt, preferably 0.5 to 20% by wt, in particular 1.0 to 15.0% by wt, relative to the total weight of the preparations, in order to make available cosmetic preparations that protect the hair or the skin from the entire region of ultraviolet radiation. They can also serve as sunscreens for the hair or the skin.

Advantageous UV-A filter substances in the sense of the present invention are dibenzoylmethane derivatives, in particular 4-(tert-butyl)-4′-methoxydibenzoylmethane (CAS No. 70356-09-1), which is sold by Givaudan under the trademark Parsol® 1789 and by Merck under the trade name Eusolex® 9020.

Other advantageous UV-A filter substances are phenylene-1,4-bis(2-benzimidazyl)-3,3′-5,5′-tetrasulfonic acid 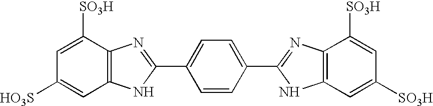
and its salts, in particular the corresponding sodium, potassium, or triethanol ammonium salts, in particular the phenylene-1,4-bis(2-benzimidazyl)-3,3′-5,5′-tetrasulfonic acid bis-sodium salt 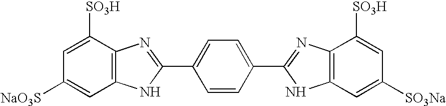
with the INCI name bisimidazylate, which is obtainable for example from Haarmann & Reimer under the trade name Neo Heliopan AP.

1,4-Di(2-oxo-10-sulfo-3-bornylidenemethyl)benzene and its salts (particularly the corresponding 10-sulfato compounds, in particular the corresponding sodium-, potassium-, or triethanol ammonium salt), which is also called benzene-1,4-di(2-oxo-3-bornylidenemethyl-10-sulfonic acid) and is characterized by the following structure: 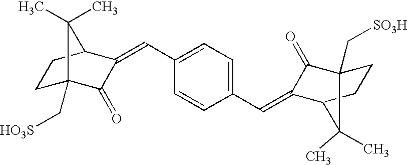
are also advantageous.

Advantageous UV filter substances in the sense of the present invention are furthermore so-called broadband filters, i.e. filter substances that absorb both UV-A and UV-B radiation.

Advantageous broadband filters or UV-B filter substances are for example bis-resorcinyltriazine derivatives with the following structure: 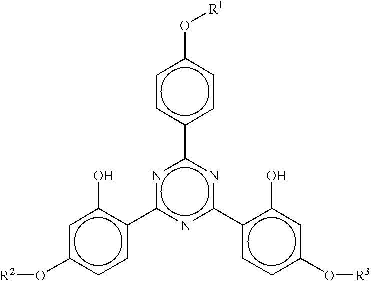
where R¹, R², and R³ independently of one another are selected from the group of branched and unbranched alkyl groups with 1 to 10 carbon atoms or represent a single hydrogen atom. 2,4-Bis{[4-(2-ethylhexyloxy)-2-hydroxy]phenyl}-6-(4-methoxyphenyl)-1,3,5-triazine (INCI: Aniso Triazin), which is obtainable from CIBA-Chemikalien GmbH under the trade name Tinosorb® S, and 4,4′,4″-(1,3,5)-triazine-2,4,6-triyltriimino)-tris-benzoic acid-tris(2-ethylhexyl ester), synonym: 2,4,6-tris[anilino(p-carbo-2′-ethyl-1′-hexyloxy)]-1,3,5-triazine (INCI: Octyl Triazone), which is sold by BASF Aktiengesellschaft under the generic name UVINUL® T 150, are particularly preferred.

Other UV filter substances that feature the structure 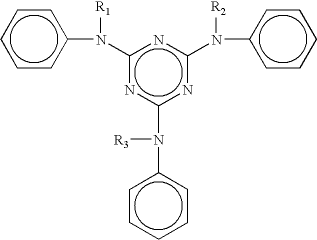
are also advantageous UV filter substances in the sense of the present invention, for example the s-triazine derivatives described in European unexamined patent application EP 570 838 A1, whose chemical structure is given by the generic formula 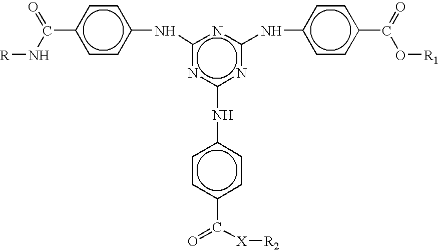
where
R represents a branched or unbranched (C₁-C₁₈)-alkyl radical, a (C₅-C₁₂)-cycloalkyl radical, optionally substituted with one or more (C₁-C₄)-alkyl groups,
X represents an oxygen atom or an NH group,
R₁ denotes a branched or unbranched (C₁-C₁₈)-alkyl radical, a (C₅-C₁₂)-cycloalkyl radical, optionally substituted with one or more (C₁-C₄)-alkyl groups, or a hydrogen atom, an alkali metal atom, an ammonium group, or a group of the formula 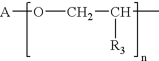
   in which
   A represents a branched or unbranched (C₁-C₁₈)-alkyl radical, a (C₅-C₁₂)-cycloalkyl- or aryl radical, optionally substituted with one or more (C₁-C₄)-alkyl groups,
   R₃ represents a hydrogen atom or a methyl group,
   n represents a number from 1 to 10,
R₂ represents a branched or unbranched (C₁-C₁₈)-alkyl radical, a (C₅-C₁₂)-cycloalkyl radical, optionally substituted with one or more (C₁-C₄)-alkyl groups, when X represents the NH group, and denotes a branched or unbranched (C₁-C₁₈)-alkyl radical, a (C₅-C₁₂)-cycloalkyl radical, optionally substituted with one or more (C₁-C₄)-alkyl groups, or a hydrogen atom, an alkali metal atom, an ammonium group, or a group of the formula 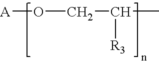
   in which
   A represents a branched or unbranched (C₁-C₁₈)-alkyl radical, a (C₅-C₁₂)-cycloalkyl- or aryl radical, optionally substituted with one or more (C₁-C₄)-alkyl groups,
   R₃ represents a hydrogen atom or a methyl group,
   n represents a number from 1 to 10,
   when X represents an oxygen atom.

A particularly advantageous UV filter substance in the sense of the present invention is furthermore an unsymmetrically substituted s-triazine whose chemical structure is shown by the formula 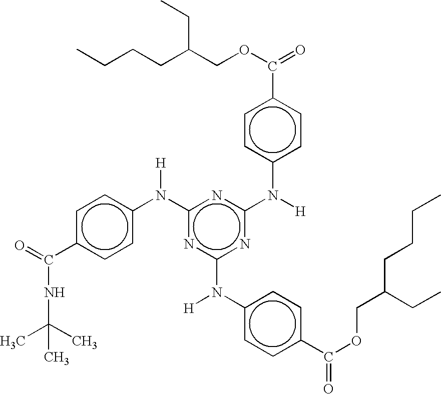
which is also called dioctylbutylamidotriazone (INCI: Dioctylbutamidotriazone) below and is obtainable from Sigma 3V under the trade name UVASORB HEB.

Also in European unexamined patent application 775 698 bis-resorcinyltriazine derivatives to be used advantageously are described whose chemical structure is shown by the generic formula 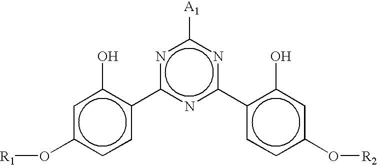
where R₁, R₂, and A₁ represent a great variety of organic radicals.

The following are furthermore advantageous in the sense of the present invention: 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)phenyl-amino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]-phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2“-methylpropenyloxy)-2-hydroxy]phenyl}-6-(4-methoxyphenyl)-1,3,5-triazine, and 2,4-bis{[4-(1′,1′,1′,3′,3′,5′,5′-heptamethylsiloxy-2″-methylpropyloxy)-2-hydroxy]phenyl}-6-(4-methoxyphenyl)-1,3,5-triazine.

An advantageous broadband filter in the sense of the present invention is 2,2′-methylene-bis(6-(2H-benzotriazol-2-yl)-4-(1,1,3,3-tetramethylbutyl)phenol) [INCI: Bisoctyltriazol], which is characterized by the chemical structural formula 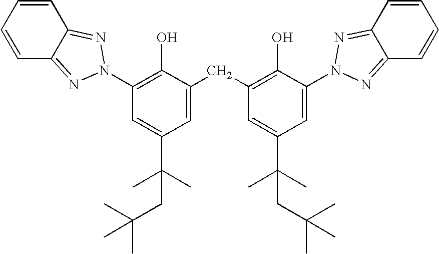
and is obtainable from CIBA Chemikalien GmbH under the trade name Tinosorb® M.

An advantageous broadband filter in the sense of the present invention is furthermore 2-(2H-benzotriazol-2-yl)4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is characterized by the chemical structural formula

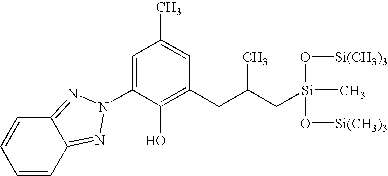

The UV-B filter can be oil-soluble or water-soluble. Advantageous oil-soluble UV-B filter substances are e.g.:
3-benzylidenecamphor derivatives, preferably 3-(4-methylbenzylidene)camphor, 3-benzylidenecamphor;
4-aminobenzoic acid derivatives, preferably 4-(dimethylamino)benzoic acid(2-ethylhexyl)ester, 4-(dimethylamino)benzoic acid amyl ester;
2,4,6-trianilino(p-carbo-2′-ethyl-1′-hexyloxy)-1,3,5-triazine;
esters of benzalmalonic acid, preferably 4-methoxybenzalmalonic acid di(2-ethylhexyl)ester;
esters of cinnamic acid, preferably 4-methoxycinnamic acid(2-ethylhexyl)ester, 4-methoxycinnamic acid isopentyl ester;
derivatives of benzophenone, preferably 2-hydroxy4-methoxybenzophenone, 2-hydroxy-4-methoxy-4′-methylbenzophenone, 2,2′-dihydroxy-4-methoxybenzophenone
as well as UV filters bound to polymers.

Advantageous water-soluble UV-B filter substances are e.g.:
salts of 2-phenylbenzimidazole-5-sulfonic acid, such as their sodium-, potassium-, or their triethanol ammonium salt, as well as the sulfonic acid itself;
sulfonic acid derivatives of 3-benzylidenecamphor, such as e.g. 4-(2-oxo-3-bornylidenemethyl)benzenesulfonic acid, 2-methyl-5-(2-oxo-3-bornylidenemethyl)sulfonic acid and their salts.

A further light protection filter substance advantageously to be used according to the invention is ethylhexyl-2-cyano-3,3-diphenyl acrylate (Octocrylene), which is obtainable from BASF under the name Uvinul® and is characterized by the following structure 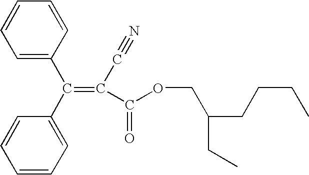
It can also be of considerable advantage to use polymer-bound or polymer UV filter substances in preparations to be used according to the present invention, in particular those as described in WO-A-92/20690.

If applicable, it can furthermore be advantageous to incorporate further UV-A and/or UV-B filters according to the invention in cosmetic or dermatological preparations, for example certain salicylic acid derivatives such as 4-isopropylbenzyl salicylate, 2-ethylhexyl salicylate (=octyl salicylate), homomenthyl salicylate.

The list of the named UV filters that can be used in the sense of the present invention is of course not intended to be limiting.

Cosmetic and dermatological preparations according to the invention advantageously contain in addition inorganic pigments based on metal oxides and/or based on other metal compounds that are poorly soluble or insoluble in water, in particular the oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, as well as mixtures of such oxides. It is particularly preferred for these to be pigments based on TiO₂.

It is particularly advantageous in the sense of the present invention, although not mandatory, if the inorganic pigments are present in hydrophobic form, i.e., that they are treated so that their surfaces are water-repellent. This surface treatment can comprise the pigments being provided with a thin hydrophobic layer according to per se known methods.

Such a method for example exists in that the hydrophobic surface layer is produced after a reaction according to
n TiO₂+m (RO)₃Si—R′→n TiO₂ (surface).
n and m thereby are stoichiometric parameters that can be used as desired, R and R′ are the desired organic radicals. For example hydrophobized pigments represented in analogy to DE-OS 33 14 742 are advantageous.

Advantageous TiO₂ pigments are for example obtainable from the TAYCA company under the trade name MT 100 T, furthermore M 160 from the Kemira company and T 805 from the Degussa company.

Preparations according to the invention, especially when crystalline or microcrystalline solids, for example inorganic micropigments, are to be incorporated into the preparations according to the invention, can also contain anionic, nonionic, and/or amphoteric surfactants. Surfactants are amphiphilic substances that can dissolve organic nonpolar substances in water.

The hydrophilic portions of a surfactant molecule are usually polar functional groups, for example —COO⁻, —OSO₃²⁻, —SO₃⁻, whereas the hydrophobic parts as a rule represent nonpolar hydrocarbon radicals. Surfactants are generally classified according to the type and charge of the hydrophilic molecule part. Four groups can be distinguished hereby:
anionic surfactants
cationic surfactants
amphoteric surfactants and
nonionic surfactants.

Anionic surfactants feature as a rule carboxylate-, sulfate-, or sulfonate groups as functional groups. In aqueous solution they form negatively charged organic ions in an acidic or neutral medium. Cationic surfactants are almost exclusively characterized by the presence of a quaternary ammonium group. In aqueous solution they form positively charged organic ions in an acidic or neutral medium. Amphoteric surfactants contain both anionic and cationic groups and therefore behave in aqueous solution like anionic or cationic surfactants, depending on the pH. In a strongly acidic medium they have a positive charge and in an alkaline medium they have a negative charge. In the neutral pH range, on the other hand, they are zwitterionic, as the following example is to illustrate:
RNH₂⁺CH₂CH₂COOH X⁻ (at pH=2) X⁻=any anion, e.g. Cl⁻
RNH₂⁺CH₂CH₂COO (at pH=7)
RNHCH₂CH₂COO⁻B⁺ (at pH=12) B⁺=any cation, e.g. Na⁺

Polyether chains are typical of nonionic surfactants. Nonionic surfactants do not form any ions in an aqueous medium.

A. Anionic Surfactants

Anionic surfactants to be used advantageously are acylamino acids (and their salts), such as
1. acyl glutamates, for example sodium acyl glutamate, Di-TEA-palmitoyl aspartate, and sodium caprylic/capric glutamate,
2. acyl peptides, for example palmitoyl-hydrolyzed milk protein, sodium cocoyl-hydrolyzed soybean protein, and sodium/potassium cocoyl-hydrolyzed collagen,
3. sarcosinates, for example myristoyl sarcosine, TEA-lauroyl sarcosinate, sodium lauroyl sarcosinate, and sodium cocoyl sarcosinate,
4. taurates, for example sodium lauroyl taurate and sodium methyl cocoyl taurate,
5. acyl lactylates, lauroyl lactylate, octanoyl lactylate,
6. alaninates
   carboxylic acids and derivatives, such as
1. carboxylic acids, for example lauric acid, aluminum stearate, magnesium alkanolate, and zinc undecylenate,
2. ester carboxylic acids, for example calcium stearoyl lactylate, laureth-6 citrate, and sodium PEG4 lauramidocarboxyate,
3. ether carboxylic acids, for example sodium laureth-13 carboxylate and sodium PEG-6 cocamide carboxylate,
   phosphoric acid esters and salts, such as for example DEA-oleth-10-phosphate and dilaureth-4-phosphate,
   sulfonic acids and salts, such as
1. acyl isethionates, e.g. sodium/ammonium cocoyl isethionate,
2. alkylaryl sulfonates,
3. alkyl sulfonates, for example sodium cocosmonoglyceride sulfate, sodium (C₁₂₋₁₄)-olefin sulfonate, sodium lauryl sulfoacetate, and magnesium PEG-3 cocamidosulfate,
4. sulfosuccinates, for example dioctylsodium sulfosuccinate, disodium laurethsulfosuccinate, disodium laurylsulfosuccinate, and disodium undecyleneamido MEA-sulfosuccinate,
   as well as sulfuric acid esters such as
1. alkyl ether sulfate, for example sodium-, ammonium-, magnesium-, MIPA-, TIPA-laureth sulfate, sodium myreth sulfate, and sodium (C₁₂₋₁₃)-pareth sulfate,
2. alkyl sulfates, for example sodium-, ammonium-, and TEA-lauryl sulfate.

B. Cationic Surfactants

Cationic surfactants to be used advantageously are
1. alkylamines,
2. alkylimidazoles,
3. ethoxylated amines, and
4. quaternary surfactants.
5. esterquats

Quaternary surfactants contain at least one N atom that is covalently bound to 4 alkyl- or aryl groups. This leads, independent of the pH, to a positive charge. Alkylbetaine, alkylamidopropylbetaine, and alkylamidopropylhydroxysulfaine are advantageous. The cationic surfactants used according to the invention can furthermore be preferably selected from the group of quaternary ammonium compounds, in particular benzyltrialkylammonium chlorides or -bromides, such as for example benzyldimethylstearylammonium chloride, furthermore alkyltrialkylammonium salts, for example cetyltrimethylammonium chloride or -bromide, alkyldimethylhydroxyethylammonium chlorides or -bromides, dialkyldimethylammonium chlorides or -bromides, alkylamidoethyltrimethylammonium ether sulfates, alkylpyridinium salts, for example lauryl- or cetylpyrimidinium chloride, imidazoline derivatives and compounds with cationic character such as amine oxides, for example alkyldimethylamine oxides or alkylaminoethyldimethylamine oxides. In particular, cetyltrimethylammonium salts are to be used advantageously.

C. Amphoteric Surfactants

Amphoteric surfactants to be used advantageously are
1. acyl/dialkylethylenediamine, for example sodium acylamphoacetate, disodium acylamphodipropionate, disodium alkylamphodiacetate, sodium acylamphohydroxypropylsulfonate, disodium acylamphodiacetate, and sodium acylamphopropionate,
2. N-alkylamino acids, for example aminopropylalkylglutamide, alkylaminopropionic acid, sodium alkylimidodipropionate, and lauroamphocarboxyglycinate.

D. Nonionic Surfactants

Nonionic surfactants to be used advantageously are
1. alcohols,
2. alkanol amides, such as cocamides MEA/ DEA/MIPA,
3. amine oxides, such as cocoamidopropylamine oxide,
4. esters formed by esterification of carboxylic acids with ethylene oxide, glycerol, sorbitan, or other alcohols,
5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers, and alkylpolyglycosides such as laurylglucoside, decylglycoside, and cocoglycoside,
6. sucrose esters, -ethers,
7. polyglycerol esters, diglycerol esters, monoglycerol esters,
8. methylglucose esters, esters of hydroxy acids.

The use of a combination of anionic and/or amphoteric surfactants with one or more nonionic surfactants is furthermore advantageous.

The surface-active substance can be present in the preparations according to the invention in a concentration between 1 and 95% by wt, relative to the total weight of the preparations.

The lipid phase of the cosmetic or dermatological emulsions according to the invention can be advantageously selected from the following substance group:
mineral oils, mineral waxes;
oils, such as triglycerides of capric or caprylic acid, furthermore natural oils such as e.g. castor oil;
fats, waxes and other natural and synthetic adipoids, preferably esters of fatty acids with alcohols of low C number, e.g. with isopropanol, propylene glycol or glycerol, or esters of fatty alcohols with alkanoic acids of low C number or with fatty acids;
alkyl benzoates;
silicone oils such as dimethylpolysiloxanes, diethylpolysiloxanes, diphenylpolysiloxanes, and mixed forms thereof.

The oil phase of the emulsions of the present invention is advantageously selected from the group of esters of saturated and/or unsaturated, branched and/or unbranched alkanecarboxylic acids of a chain length from 3 to 30 C atoms and saturated and/or unsaturated, branched and/or unbranched alcohols of a chain length from 3 to 30 C atoms, from the group of the esters of aromatic carboxylic acids and saturated and/or unsaturated, branched and/or unbranched alcohols of a chain length from 3 to 30 C atoms. Such ester oils can then be advantageously selected from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, as well as synthetic, semisynthetic, and natural mixtures of such esters, e.g. jojoba oil.

Furthermore the oil phase can be advantageously selected from the group of the branched and unbranched hydrocarbons and hydrocarbon waxes, the silicone oils, the dialkyl ethers, the group of the saturated or unsaturated, branched or unbranched alcohols, as well as of the fatty acid triglycerides, namely the triglycerol esters of saturated and/or unsaturated, branched and/or unbranched alkanecarboxylic acids of a chain length from 8 to 24, in particular 12 to 18, C atoms. The fatty acid triglycerides can for example be advantageously selected from the group of the synthetic, semisynthetic, and natural oils, e.g. olive oil, sunflower oil, soybean oil, peanut oil, rapeseed oil, almond oil, palm oil, coconut oil, palm kernel oil, and the like.

Any desired mixtures of such oil and wax components are also to be used advantageously in the sense of the present invention. If applicable, it can also be advantageous to use waxes, for example cetyl palmitate, as the single lipid component of the oil phase.

The oil phase is advantageously selected from the group 2-ethylhexyl isostearate, octyldodecanol, isotridecyl isononanoate, isoeicosane, 2-ethylhexyl cocoate, (C₁₂₋₁₅)-alkyl benzoate, capric-caprylic acid triglyceride, dicaprylyl ether.

Mixtures of (C₁₂₋₁₅)-alkyl benzoate and 2-ethylhexyl isostearate, mixtures of (C₁₂₋₁₅)-alkyl benzoate and isotridecyl isononanoate, as well as mixtures of (C₁₂₋₁₅)-alkyl benzoate, 2-ethylhexyl isostearate, and isotridecyl isononanoate, are particularly advantageous.

Of the hydrocarbons, paraffin oil, squalane, and squalene are to be used advantageously in the sense of the present invention.

Advantageously the oil phase can furthermore feature a content of cyclic or linear silicone oils or can be composed completely of such oils, whereby however it is preferred to use an additional content of other oil phase components in addition to the silicone oil or silicone oils. Such silicones or silicone oils can be present as monomers that as a rule are characterized by structural elements as follows:

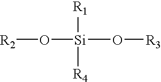

The linear silicones with several siloxyl units to be used advantageously according to the invention are generally characterized by structural elements as follows: 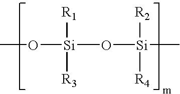
where the silicon atoms can be substituted by the same or different alkyl radicals and/or aryl radicals, which here are represented generalizingly by the radicals R₁-R₄ (that is to say that the number of different radicals is not necessarily limited to up to 4). m can thereby take on values of 2-200,000.

Cyclic silicones to be used advantageously according to the invention are generally characterized by structural elements as follows: 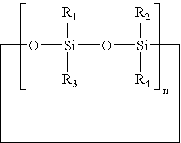
where the silicon atoms can be substituted by the same or different alkyl radicals and/or aryl radicals, which here are represented generalizingly by the radicals R₁-R₄ (that is to say that the number of different radicals is not necessarily limited to up to 4). n can thereby take on values of 3/2 to 20. Fractional values for n take into consideration the fact that odd-numbered numbers of siloxyl groups can be present in the ring.

Advantageously cyclomethicone (e.g. decamethylcyclopentasiloxane) is used as the silicone oil to be used according to the invention. However, other silicone oils are also to be used advantageously in the sense of the present invention, for example undecamethylcyclotrisiloxane, polydimethylsiloxane, poly(methylphenylsiloxane), cetyl dimethicone, behenoxy dimethicone.

Mixtures of cyclomethicone and isotridecyl isononanoate, as well as those of cyclomethicone and 2-ethylhexyl isostearate, are furthermore advantageous.

However, it is also advantageous to select silicone oils of similar constitution to the compounds named above whose organic side chains are derivatized, for example polyethoxylated and/or polypropoxylated. These include for example polysiloxane/polyalkylpolyether copolymers such as the cetyl/dimethicone copolyol, the (cetyl dimethicone copolyol (and) polyglyceryl-4-isostearate (and) hexyl laurate).

Furthermore mixtures of cyclomethicone and isotridecyl isononanoate, of cyclomethicone and 2-ethylhexyl isostearate, are particularly advantageous.

If applicable, the aqueous phase of the preparations according to the invention advantageously contains alcohols, diols or polyols of low C number, as well as their ethers, preferably ethanol, isopropanol, propylene glycol, glycerol, ethylene glycol, ethylene glycol monoethyl- or -monobutyl ether, propylene glycol monomethyl, -monoethyl-, or -monobutyl ether, diethylene glycol monomethyl- or -monoethyl ether and analogous products, furthermore alcohols of low C number, e.g. ethanol, isopropanol, 1,2-propanediol, glycerol, as well as in particular one or more thickening agents, which can advantageously be selected from the group silicon dioxide, aluminum silicates.

Preparations according to the invention present as emulsions contain in particular advantageously one or more hydrocolloids. These hydrocolloids can advantageously be selected from the group of the gums, polysaccharides, cellulose derivatives, sheet silicates, polyacrylates, and/or other polymers.

Preparations according to the invention present as hydrogels contain one or more hydrocolloids. These hydrocolloids can advantageously be selected from the above-mentioned group.

The gums are considered to include plant juices or tree saps that harden in air and form resins or extracts of aquatic plants. In the sense of the present invention, the following can advantageously be selected from this group: for example gum arabic, carob gum, tragacanth, karaya gum, guar gum, pectin, gellan gum, carrageenan, agar, algine, chondrus, xanthan gum.

Moreover the use of derivatized gums such as e.g. hydroxypropyl guar (Jaguar® HP 8) is advantageous.

The polysaccharides and -derivatives include e.g. hyaluronic acid, chitin and chitosan, chondroitin sulfates, starch and starch derivatives.

The cellulose derivatives include e.g. methylcellulose, carboxymethylcellulose, hydroxyethylcellulose, hydroxypropylmethylcellulose.

The sheet silicates include naturally occurring and synthetic clays such as e.g. montmorillonite, bentonite, hectorite, laponite, magnesium aluminum silicates such as Veegum®. These can be used as such or in modified form such as e.g. Stearylalkonium Hectorite.

Moreover silica gels can also be used advantageously.

The polyacrylates include e.g. the Goodrich company Carbopol types (Carbopol 980, 981, 1382, 5984, 2984, EDT 2001 or Pemulen TR2).

The polymers include e.g. polyacrylamides (Seppigel 305), polyvinyl alcohols, PVP, PVPNA copolymers, polyglycols.

Preparations according to the invention present as emulsions contain one or more emulsifiers. These emulsifiers can advantageously be selected from the group of the nonionic, anionic, cationic, or amphoteric emulsifiers.

The nonionic emulsifiers include
a) partial fatty acid esters and fatty acid esters of polyvalent alcohols and their ethoxylated derivatives (e.g. glyceryl monostearates, sorbitan stearates, glycerylstearyl citrates, sucrose stearates)
b) ethoxylated fatty alcohols and fatty acids
c) ethoxylated fatty amines, fatty acid amides, fatty acid alkanol amides
d) alkylphenol polyglycol ethers (e.g. Triton X)
   The anionic emulsifiers include
a) soaps (e.g. sodium stearate)
b) fatty alcohol sulfates
c) mono-, di-, and trialkylphosphoric esters and their ethoxylates

The cationic emulsifiers include
a) quaternary ammonium compounds with a long-chain aliphatic radical, e.g. Distearyldimonium Chloride

The amphoteric emulsifiers include
a) alkylamininoalkanecarboxylic acids
b) betaines, sulfobetaines
c) imidazoline derivatives

Moreover there are naturally occurring emulsifiers, including beeswax, wool fat, lecithin, and sterols.

O/W emulsifiers can for example be selected advantageously from the group of the polyethoxylated or polypropoxylated or polyethoxylated and polypropoxylated products, e.g.:
the fatty alcohol ethoxylates
the ethoxylated wool fat alcohols
the polyethylene glycol ethers of the general formula R—O—(—CH₂—CH₂—O—)ₙ—R′
the fatty acid ethoxylates of the general formula R—COO—(—CH₂—CH₂—O—)ₙ—H
the etherified fatty acid ethoxylates of the general formula R—COO—(—CH₂—CH₂—O—)ₙ—R′
the esterified fatty acid ethoxylates of the general formula R—COO—(—CH₂—CH₂—O—)ₙ—C(O)—R′
the polyethylene glycol glycerol fatty acid esters
the ethoxylated sorbitan esters
the cholesterol ethoxylates
the ethoxylated triglycerides
the alkyl ether carboxylic acids of the general formula R—O—(—CH₂—CH₂—O—)ₙ—CH₂—COOH nd n represent[s] a number from 5 to 30
the polyoxyethylene sorbitol fatty acid esters
the alkyl ether sulfates of the general formula R—O—(—CH₂—CH₂—O—)ₙ—SO₃—H
the fatty alcohol propoxylates of the general formula R—O—(—CH₂—CH(CH₃)—O—)ₙ—H
the polypropylene glycol ethers of the general formula R—O—(—CH₂—CH(CH₃)—O—)ₙ—R′
the propoxylated wool wax alcohols
the etherified fatty acid propoxylates R—COO—(—CH₂—CH(CH₃)—O—)ₙ—R′
the esterified fatty acid propoxylates of the general formula R—COO—(—CH₂—CH(CH₃)—O—)ₙ—C(O)—R′
the fatty acid propoxylates of the general formula R—COO—(—CH₂—CH(CH₃)—O—)ₙ—H
the polypropylene glycol glycerol fatty acid esters
the propoxylated sorbitan esters
the cholesterol propoxylates
the propoxylated triglycerides
the alkyl ether carboxylic acids of the general formula R—O—(—CH₂—CH(CH₃)—O—)ₙ—CH₂—COOH
the alkyl ether sulfates or the acids on which these sulfates are based of the general formula R—O—(—CH₂—CH(CH₃)—O—)ₙ—SO₃—H
the fatty alcohol ethoxylates/propoxylates of the general formula R—O—Xₙ—Yₘ—H
the polypropylene glycol ethers of the general formula R—O—Xₙ—Yₘ—R′
the etherified fatty acid propoxylates of the general formula R—COO—Xₙ—Yₘ—R′
the fatty acid ethoxylates/propoxylates of the general formula R—COO—Xₙ—Yₘ—H.

According to the invention the polyethoxylated or polypropoxylated or polyethoxylated and polypropoxylated O/W emulsifiers are particularly advantageously selected from the group of the substances with HLB values of 11-18, quite particularly advantageously with HLB values of 14.5-15.5, in so far as the O/W emulsifiers feature saturated radicals R and R′. If the O/W emulsifiers feature unsaturated radicals R and/or R′, or if isoalkyl derivatives are present, the preferred HLB value of such emulsifiers can also lie lower or higher.

It is advantageous to select the fatty alcohol ethoxylates from the group of the ethoxylated stearyl alcohols, cetyl alcohols, cetylstearyl alcohols (cetearyl alcohols). The following are preferred in particular:
polyethylene glycol(13)stearyl ether (steareth-13), polyethylene glycol(14)stearyl ether (steareth-14), polyethylene glycol(15)stearyl ether (steareth-15), polyethylene glycol(16)stearyl ether (steareth-16), polyethylene glycol(17)stearyl ether (steareth-17), polyethylene glycol(18)stearyl ether (steareth-18), polyethylene glycol(19)stearyl ether (steareth-19), polyethylene glycol(20)stearyl ether (steareth-20),
polyethylene glycol(12)isostearyl ether (isosteareth-12), polyethylene glycol(13)isostearyl ether (isosteareth-13), polyethylene glycol(14)isostearyl ether (isosteareth-14), polyethylene glycol(15)isostearyl ether (isosteareth-15), polyethylene glycol(16)isostearyl ether (isosteareth-16), polyethylene glycol(17)isostearyl ether (isosteareth-17), polyethylene glycol(18)isostearyl ether (isosteareth-18), polyethylene glycol(19)isostearyl ether (isosteareth-19), polyethylene glycol(20)isostearyl ether (isosteareth-20),
polyethylene glycol(13)cetyl ether (ceteth-13), polyethylene glycol(14)cetyl ether (ceteth-14), polyethylene glycol(15)cetyl ether (ceteth-15), polyethylene glycol(16)cetyl ether (ceteth-16), polyethylene glycol(17)cetyl ether (ceteth-17), polyethylene glycol(18)cetyl ether (ceteth-18), polyethylene glycol(19)cetyl ether (ceteth-19), polyethylene glycol(20)cetyl ether (ceteth-20),
polyethylene glycol(13)isocetyl ether (isoceteth-13), polyethylene glycol(14)isocetyl ether (isoceteth-14), polyethylene glycol(15)isocetyl ether (isoceteth-15), polyethylene glycol(16)isocetyl ether (isoceteth-16), polyethylene glycol(17)isocetyl ether (isoceteth-17), polyethylene glycol(18)isocetyl ether (isoceteth-18), polyethylene glycol(19)isocetyl ether (isoceteth-19), polyethylene glycol(20)isocetyl ether (isoceteth-20),
polyethylene glycol(12)oleyl ether (oleth-12), polyethylene glycol(13)oleyl ether (oleth-13), polyethylene glycol(14)oleyl ether (oleth-14), polyethylene glycol(15)oleyl ether (oleth-15),
polyethylene glycol(12)lauryl ether (laureth-12), polyethylene glycol(12)isolauryl ether (isolaureth-12),
polyethylene glycol(13)cetylstearyl ether (ceteareth-13), polyethylene glycol(14)cetylstearyl ether (ceteareth-14), polyethylene glycol(15)cetylstearyl ether (ceteareth-15), polyethylene glycol(16)cetylstearyl ether (ceteareth-16), polyethylene glycol(17)cetylstearyl ether (ceteareth-17), polyethylene glycol(18)cetylstearyl ether (ceteareth-18), polyethylene glycol(19)cetylstearyl ether (ceteareth-19), polyethylene glycol(20)cetylstearyl ether (ceteareth-20).

It is furthermore advantageous to select the fatty acid ethoxylates from the following group:
polyethylene glycol(20)stearate, polyethylene glycol(21 )stearate, polyethylene glycol(22)stearate, polyethylene glycol(23)stearate, polyethylene glycol(24)stearate, polyethylene glycol(25)stearate,
polyethylene glycol(12)isostearate, polyethylene glycol(13)isostearate, polyethylene glycol(14)isostearate, polyethylene glycol(15)isostearate, polyethylene glycol(16)isostearate, polyethylene glycol(17)isostearate, polyethylene glycol (18)isostearate, polyethylene glycol(19)isostearate, polyethylene glycol(20)isostearate, polyethylene glycol(21 )isostearate, polyethylene glycol(22)isostearate, polyethylene glycol(23)isostearate, polyethylene glycol(24)isostearate, polyethylene glycol(25)isostearate,
polyethylene glycol(12)oleate, polyethylene glycol(13)oleate, polyethylene glycol(14)oleate, polyethylene glycol(15)oleate, polyethylene glycol(16)oleate, polyethylene glycol(17)oleate, polyethylene glycol(18)oleate, polyethylene glycol(19)oleate, polyethylene glycol(20)oleate.

As an ethoxylated alkyl ether carboxylic acid or its salt, sodium laureth-11-carboxylate can be used advantageously.

As an alkyl ether sulfate, sodium laureth 14 sulfate can be used advantageously.

As an ethoxylated cholesterol derivative, polyethylene glycol(30)cholesteryl ether can be used advantageously. Polyethylene glycol(25) soybean sterol has also proved good.

As ethoxylated triglycerides, the polyethylene glycol(60) evening primrose glycerides can be used advantageously.

Moreover it is advantageous to select the polyethylene glycol glycerol fatty acid esters from the group polyethylene glycol(20)glyceryl laurate, polyethylene glycol(21)glyceryl laurate, polyethylene glycol(22)glyceryl laurate, polyethylene glycol(23)glyceryl laurate, polyethylene glycol(6)glyceryl caprate/caprylate, polyethylene glycol(20)glyceryl oleate, polyethylene glycol(20)glyceryl isostearate, polyethylene glycol(18)glyceryl oleate/cocoate.

It is likewise favorable to select the sorbitan esters from the group polyethylene glycol(20)sorbitan monolaurate, polyethylene glycol(20)sorbitan monostearate, polyethylene glycol(20)sorbitan monoisostearate, polyethylene glycol(20)sorbitan monopalmitate, polyethylene glycol(20)sorbitan monooleate.

As advantageous W/O emulsifiers, the following can be used: fatty alcohols with 8 to 30 carbon atoms, monoglycerol esters of saturated and/or unsaturated, branched and/or unbranched alkanecarboxylic acids of a chain length from 8 to 24, in particular 12 to 18, C atoms, diglycerol esters of saturated and/or unsaturated, branched and/or unbranched alkanecarboxylic acids of a chain length from 8 to 24, in particular 12 to 18, C atoms, monoglycerol ethers of saturated and/or unsaturated, branched and/or unbranched alcohols of a chain length from 8 to 24, in particular 12 to 18, C atoms, diglycerol ethers of saturated and/or unsaturated, branched and/or unbranched alcohols of a chain length from 8 to 24, in particular 12 to 18, C atoms, polypropylene glycol esters of saturated and/or unsaturated, branched and/or unbranched alkanecarboxylic acids of a chain length from 8 to 24, in particular 12 to 18, C atoms, as well as sorbitan esters of saturated and/or unsaturated, branched and/or unbranched alkanecarboxylic acids of a chain length from 8 to 24, in particular 12 to 18, C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprate, propylene glycol monolaurate, sorbitan monoisostearate, sorbitan monolaurate, sorbitan monooctanoate, sorbitan monoisooleate, saccharose distearate, cetyl alcohol, stearyl alcohol, arachidyl alcohol, behenyl alcohol, isobehenyl alcohol, selachyl alcohol, chimyl alcohol, polyethylene glycol(2)stearyl ether (steareth-2), glyceryl monolaurate, glyceryl monocaprylate, glyceryl monocaprate.

The following Examples are intended to explain the invention, but not to limit it. Unless stated otherwise, the numbers given refer to % by wt.

| | Examples of creams | | |
|---|---|---|---|
| | 1 | 2 | 3 |
| | % by wt | % by wt | % by wt |
| Glycerol | 5.00 | 8.00 | 10.00 |
| Ethyl hexyl cocoate | 5.30 | 5.30 | 5.30 |
| Cetyl alcohol | 3.00 | 3.00 | 3.00 |
| Hydrogenated coconut fatty | 3.00 | 3.00 | 3.00 |
| acid glycerides | | | |
| Butylene glycol | 3.00 | 3.00 | 3.00 |
| Stearyl alcohol | 3.00 | 3.00 | 3.00 |
| Caprylic acid/capric acid | 2.70 | 2.70 | 2.70 |
| triglycerides | | | |
| Tocopheryl acetate | 2.00 | 2.00 | 2.00 |
| Butyrospermum parkii | 2.00 | 2.00 | 2.00 |
| Tridecyl stearate + tridecyl | 2.00 | 2.00 | 2.00 |
| trimellitate + dipentaerythrityl | | | |
| hexacaprylate/hexacaprate | | | |
| Glyceryl stearate citrate | 2.00 | 2.00 | 2.00 |
| Ethyl paraben + methyl | 0.50 | 0.50 | 0.50 |
| paraben + propyl | | | |
| paraben + phenoxyethanol + | | | |
| butyl paraben + isobutyl | | | |
| paraben | | | |
| Carbomer | 0.40 | 0.40 | 0.40 |
| DMDM hydantoin | 0.278 | 0.278 | 0.278 |
| NaOH | 0.18 | 0.18 | 0.18 |
| Carnitine | 0.50 | 1.00 | 2.00 |
| Soybean isoflavonoids | 0.10 | 0.50 | 1.00 |
| Water | to 100.0 | to 100.00 | to 100.00 |

| | Examples of creams | | |
|---|---|---|---|
| | 4 | 5 | 6 |
| | % by wt | % by wt | % by wt |
| Glycerol | 8.00 | 10.00 | 12.00 |
| Hydrogenated coconut fatty | 5.00 | 5.00 | 5.00 |
| acid glycerides | | | |
| Stearyl alcohol | 3.50 | 3.50 | 3.50 |
| Stearic acid | 3.00 | 3.00 | 3.00 |
| Paraffinum liquidum | 3.00 | 3.00 | 3.00 |
| Cetyl alcohol | 1.50 | 1.50 | — |
| Trisodium EDTA | 1.00 | 1.00 | 1.00 |
| Dimethicone | — | 1.00 | 1.00 |
| Aluminum/starch octenyl | 1.00 | 1.00 | 1.00 |
| succinate | | | |
| Phenoxyethanol | 0.80 | 0.80 | 0.80 |
| Glyceryl stearate | 0.50 | 0.50 | 0.50 |
| Sorbitan stearate | 0.50 | 0.50 | 0.50 |
| PEG-1 stearate | 0.50 | 0.50 | 0.50 |
| Methyl paraben | 0.40 | 0.40 | 0.40 |
| Carbomer | 0.20 | 0.20 | 0.20 |
| Propyl paraben | 0.15 | 0.15 | 0.15 |
| Carnitine | 0.50 | 1.00 | 2.00 |
| Soybean isoflavonoids | 0.50 | 1.00 | 2.00 |
| Perfume | 0.03 | 0.03 | 0.03 |
| Water | to 100.0 | to 100.00 | to 100.00 |

| | Examples of creams | | |
|---|---|---|---|
| | 7 | 8 | 9 |
| | % by wt | % by wt | % by wt |
| Glycerol | 6.00 | 8.00 | 10.00 |
| Cetyl alcohol | 4.00 | 4.00 | 4.00 |
| Caprylic acid/capric acid | 3.10 | 3.10 | 3.10 |
| triglycerides | | | |
| Paraffinum liquidum | 3.00 | 5.00 | 6.00 |
| Water + denat. alcohol | 3.00 | 3.00 | 3.00 |
| Cyclomethicone | — | 2.90 | 2.90 |
| Dimethicone | 2.80 | — | 2.80 |
| Glyceryl stearate | 2.70 | 2.70 | 2.70 |
| PEG-40 stearate | 1.30 | 1.30 | 1.30 |
| Carnitine | 0.50 | 1.00 | 2.00 |
| Soybean isoflavonoids | 1.00 | 0.50 | 0.10 |
| Ethyl paraben + methyl | 0.50 | 0.50 | 0.50 |
| paraben + propyl | | | |
| paraben + phenoxyethanol + | | | |
| butylparaben + isobutyl | | | |
| paraben | | | |
| Methyl paraben | 0.20 | 0.20 | 0.20 |
| Carbomer | 0.20 | 0.20 | 0.20 |
| Perfume | 0.15 | 0.15 | 0.15 |
| Propyl paraben | 0.07 | 0.07 | 0.07 |
| NaOH | 0.04 | 0.04 | 0.04 |
| Water | to 100.0 | to 100.00 | to 100.00 |

| | Examples of lotions | | |
|---|---|---|---|
| | 10 | 11 | 12 |
| | % by wt | % by wt | % by wt |
| Glycerol | 6.00 | 8.00 | 12.00 |
| Cetyl palmitate | 10.00 | 10.00 | 10.00 |
| Paraffinum liquidum | 8.00 | 8.00 | 8.00 |
| Cetyl alcohol | 3.00 | 2.00 | — |
| Stearyl alcohol | — | 1.00 | 3.00 |
| Cyclomethicone | 3.00 | 3.00 | 3.00 |
| Sorbitan stearate | 2.00 | 2.00 | 2.00 |
| Aluminum/starch octenyl | 1.50 | — | 1.50 |
| succinate | | | |
| Phenoxyethanol | 0.80 | 0.80 | 0.80 |
| Methyl paraben | 0.30 | 0.30 | 0.40 |
| Carbomer | 0.25 | 0.25 | 0.25 |
| Carnitine | 0.50 | 1.00 | 2.00 |
| Soybean isoflavones | 1.00 | 0.50 | 1.00 |
| Propyl paraben | 0.10 | 0.10 | — |
| NaOH | 0.03 | 0.03 | 0.03 |
| Water | to 100.0 | to 100.00 | to 100.00 |

The present application claims priority under 35 U.S.C. §119 of German Patent Application DE 10 2004 060 314.6, filed Dec. 8, 2004, the entire disclosure whereof is expressly incorporated by reference herein.

1.-9. (canceled)

10. An active ingredient combination of
(a) one or more isoflavonoids and
(b) at least one of carnitine and one or more acylcarnitines.

11. The combination of claim 10, wherein a molar ratio component (a) component (b) is from 10:1 to 1:10.

12. The combination of claim 11, wherein the molar ratio is from 5:1 to 1:5.

13. The combination of claim 12, wherein the molar ratio is from 2:1 to 1:2.

14. The combination of claim 10, wherein component (a) comprises genistein.

15. The combination of claim 10, which further comprises one or more saponins.

16. A cosmetic or dermatological preparation which comprises
(a) one or more isoflavonoids and
(b) at least one of carnitine and one or more acylcarnitines.

17. The preparation of claim 16, wherein a molar ratio component (a) component (b) is from 10:1 to 1:10.

18. The preparation of claim 17, wherein the molar ratio is from 2:1 to 1:2.

19. The preparation of claim 17, wherein component (a) comprises genistein.

20. The preparation of claim 16, wherein the preparation comprises from 0.001% to 10% by wt. of component (a), relative to a total weight of the preparation.

21. The preparation of claim 17, wherein the preparation comprises from 0.01% to 1% by wt. of component (a), relative to a total weight of the preparation.

22. The preparation of claim 16, wherein the preparation comprises from 0.001% to 10% by wt. of component (b), relative to a total weight of the preparation.

23. The preparation of claim 17, wherein the preparation comprises from 0.01% to 1% by wt. of component (b), relative to a total weight of the preparation.

24. The preparation of claim 19, wherein the preparation comprises from 0.01% to 1% by wt. of component (a) and from 0.01% to 1% by wt. of component (b), relative to a total weight of the preparation.

25. The preparation of claim 16, wherein the preparation further comprises
(c) one or more saponins.

26. The preparation of claim 25, wherein the preparation comprises from 0.001% to 2% by wt. of component (c), relative to a total weight of the preparation.

27. The preparation of claim 26, wherein the preparation comprises from 0.02% to 0.04% by wt. of component (c).

28. The preparation of claim 16, wherein component (a) comprises an isoflavonoid-containing soybean extract which comprises from 5% to 20% by wt. of one or more saponins, relative to a total weight of the extract.

29. The preparation of claim 28, wherein the soybean extract comprises from 10% to 18% by wt. of one or more saponins.

30. A cosmetic or dermatological preparation which comprises
(a) from 0.001% to 10% by wt. of one or more isoflavonoids which comprise genistein and
(b) from 0.001% to 10% by wt. of at least one of carnitine and one or more acylcarnitines,
each relative to a total weight of the preparation, wherein a molar ratio (a) : (b) is from 10:1 to 1:10.

31. The preparation of claim 30, which further comprises from 0.001% to 2% by wt. of one or more saponins.

32. The preparation of claim 31, wherein the preparation comprises from 0.01% to 1% by wt. of the one or more isoflavonoids and from 0.01% to 1% by wt. of at least one of carnitine and one or more acylcarnitines and wherein the molar ratio (a): (b) is from 2:1 to 1:2.

33. The preparation of claim 32, which further comprises from 0.02% to 0.04% by wt. of the one or more saponins.
